(19) United States
(12) Patent Application Publication
(10) Pub. No.: US 2013/0324812 A1
(43) Pub. Date: Dec. 5, 2013

(54) CARDIAC PULSE COEFFICIENT OF VARIATION AND BREATHING MONITORING SYSTEM AND METHOD FOR EXTRACTING INFORMATION FROM THE CARDIAC PULSE

(71) Applicants: Edward C. BRAINARD, II, Marion, MA (US); Matthew I. d'ENTREMONT, Lakeview (CA)

(72) Inventors: Edward C. BRAINARD, II, Marion, MA (US); Matthew I. d'ENTREMONT, Lakeview (CA)

(73) Assignee: ATLANTIS LIMITED PARTNERSHIP, Marion, MA (US)

(21) Appl. No.: 13/485,302

(22) Filed: May 31, 2012

(51) Int. Cl. A61B 5/0205; A61B 5/024

(52) U.S. Cl. 600/324; 600/500; 600/493; 600/484; 600/479

(57) ABSTRACT

A system and method to extract and measure awareness and a breathing rate information from the cardiac pulse uses plethysmographic and oximeter sensors. The information finds applications in patient monitoring during surgery, intensive care, sleep therapy, and sleep detection in critical operations of airplanes, trucks, automobiles, trains, and in biofeedback therapy.

### BACKGROUND OF THE INVENTION

The invention provides a system and a method to extract and measure awareness and breathing rate information from the cardiac pulse using instruments based on plethysmographic and oximeter sensors. The invention uses the extracted and measured information in applications including patient monitoring during surgery, intensive care, sleep therapy, and sleep detection in critical operations of airplanes, trucks, automobiles, trains, and in biofeedback therapy.

### DESCRIPTION OF THE RELATED ART

U.S. Pat. No. 7,547,284 discloses a method of measuring human brain activity that includes the steps of simultaneously measuring pulses at two locations on a human subject that each receives blood from a different carotid artery that feeds a respective left and right hemisphere of the brain of the human subject, determining pulse characteristics from the measured pulses, and evaluating relative left and right hemisphere activity of the brain of the human subject based on the determined pulse characteristics. U.S. Pat. No. 7,547,284 discloses that the method may use dual photoplethysmograhic blood pulse sensors that measure left and right hemisphere activity by determining pulse amplitude difference and time or phase differences between the earlobes while the subject carries out various mental functions, where the data from the sensors are processed to provide a measure of brain function and the mental activity of the subject.

U.S. Published Application 2010/0305456 discloses another method for monitoring brain activity where left and right cardiac pulse signals are detected at bilateral locations on a body for a selected number of cardiac cycles, and computing apparatus computes the standard deviation of the left and right pulse signals for the selected number of cardiac cycles. The standard deviations are normalized on the computing apparatus by dividing the left and right standard deviations by the mean of the left and right pulse signals computed over the selected number of cardiac cycles to produce a left and a right Coefficient of Variation of the pulse signals. U.S. Published Application 2010/0305456 further discloses that a Bilateral Pulse Index is generated from the left and right Coefficients of Variation, where the Bilateral Pulse Index relates to brain activity.

Presently, there is a need for a reliable non-invasive monitor method/system for measuring mental activity in such applications as alertness detection.

In recent years a number of investigators have used cardiac pulse measurements to monitor mental activity. While the pulse amplitude appears to change with mental activity, it is not a reliable indicator of the level of activity.

Also, in recent years a number of investigators have used pulse measurements to assess physiological status, such as fluid volume {Cannesson, 2008 #308; McGrath, 2010 #302}. However, they focused mostly on pulse amplitude changes without normalization.

Sequential pulse timing difference has been employed, but all the above methods have fallen short of providing a reliable and accurate measure of alertness.

### SUMMARY OF THE INVENTION

The invention provides improvements over the prior art monitoring for measuring mental activity, by providing an improved and more reliable indicator of the level of activity and awareness of the subject.

The inventor's data from clinical trials indicates that an increase in mental activity correlates well with an increase in standard deviation of pulse amplitude. However, standard deviation and amplitude measurements are not universally useable as a patient monitor since they are patient specific being related to the heart and circulatory characteristics of that patient. There is a need to make a universal measurement between subjects. By normalizing standard deviation measurements, the derived parameter can be applied to all subjects using a universal scale. Normalization is accomplished by dividing the standard deviation during a selected sampling period by the mean for that same time period. Statistically this provides the Coefficient of Variation (CoV) expressed in percent (%).

Discovery of a reduction of CoV for a resting mind and an increase in CoV for an active mind is based on sound scientific principles.

The inventor's hypothesis for the operation of the inventive system is based on consciousness as an interrelated operation of past, present, and future cognitive areas of the brain when functioning in harmony. In other words, these areas are interconnected when awake. During unconsciousness, the interconnecting communication pathways are disconnected. Sleep produces a similar breaking of the communication pathways. Once the pathways are disconnected, the need for blood flow is minimized due to lower brain activity.

The theory of Dark Energy indicates that there is always an underlying level of activity to support necessary autonomic body functions. Thus, there is a base level for rest or sleep conditions. The inventor's studies have indicated that this base approaches a CoV of about 4.0%. The inventive monitoring system has been designed to measure well below this anticipated base level.

The inventor's hypothesis is that brain activities can be assessed by monitoring the cardiac pulse in subjects. The postulated principle is based on the fact that measurement of blood flow in areas such as the earlobes or the forehead is closely related to the blood flow to the brain through the carotid arteries. The external carotid arteries are branches of the carotid arteries. These vessels supply blood to the jaw, face, scalp, and the ears. A measurement of blood flow at the earlobes or the forehead may relate to the measurement of the flow of blood to the brain. The inventor's studies have demonstrated that there is an increase in CoV with an increase in mental activity from the resting state. Measurement at the earlobes provides a better measurement of mental activity due to lower noise at the earlobes than at the index fingers. In clinical trials the inventor has determined that bilateral measurements at the earlobes are not a suitable measure of left and right hemispheric brain activity.

The data from this study indicates that the use of Pulse CoV techniques is suitable to provide a robust, reliable and easily installed method for measuring brain activities or for sleep detection. The tests further indicated that the use of a single sensor on an earlobe or index finger could also provide a reliable measure of mental activity since there is statistically only a small difference between left and right measurements. The results should be reproducible at the forehead, ear canal and nose since the external carotid arteries also provide blood supply to these regions. The invention thus avoids the need for bilateral measurements.

The use of CoV to normalize variance data may be used with any pulse oximeter. This means that the Pulse CoV monitor technique together with the pulse oximeters may be used in other clinical environments to determine brain activities, such as in the operating room and the Intensive Care Units.

### BRIEF DESCRIPTION OF THE DRAWINGS

FIG. 1 illustrates a Pulse CoV Monitor control panel. The display shows the output from a dual channel Pulse CoV monitoring system.

FIG. 2 illustrates Coefficient of Variation during Various Mental Activity States. Bars for Letters, Shapes, Dots, and Math tests of the plotted data are for multi body locations at the right and left earlobes and index fingers. However, clinical trials have indicated that a single sensor is suitable to detect consciousness by monitoring Coefficient of Variation (CoV). Thus, bars for lower levels of consciousness are single bars.

FIG. 3a illustrates dual channel index finger data during a ten (10) minute rest experiment. The right index finger is a solid line and the left index finger data trace is a hatched line. The horizontal line at 7% Coefficient of Variation (CoV) (y-axis) indicates a value of CoV under which a subject is entering a relaxed mental state of consciousness.

FIG. 3b illustrates dual channel earlobe data during a (10) minute rest experiment. The right earlobe data is a solid line and the left earlobe data trace is a hatched line. The horizontal line at 7% Coefficient of Variation (CoV) (y-axis) indicates a value under which a subject is entering a relaxed mental state of consciousness. The earlobe trace in FIG. 3b shows less variability the FIG. 3a. Both FIG. 3a and FIG. 3b show data for the same rest experiment.

FIGS. 4a and 4b illustrate respectively a Fast Fourier Transform and Maximum Entropy Method Plot of the cardiac pulse spectrum. This data was taken with a dual channel monitor. The hatched traces are for the left earlobe. The constant trace is for the left earlobe.

FIG. 5 illustrates a continuously running 3D plot of heart rate, breathing Rate and Coefficient of Variation. Heart Rate in Beats Per Minute (BPM) is shown on the x-axis. Breathing Rate in Breaths Per Minute (BPM) is shown in the y-axis. Coefficient of Variation (CoV) in Percent (%) is shown on the Z-axis. The data points represent data monitored for 60 seconds. The data points are updated every pulse where the oldest data point is removed and the newest data point is added to the plot.

FIG. 6 is a flow chart illustrating key steps in the inventive method.

FIG. 7 diagrammatically illustrates an embodiment of the invention as applied to a subject or patient.

### DESCRIPTION OF PREFERRED EMBODIMENTS

Although the invention is not limited to any particular sensor, and may in general be applied with any appropriate plethysmographic sensor or pulse oximeter sensor.

For example, a pulse oximeter sensor such as a Masimo™ LNCS TC-1 Tip Clip Oximeter sensor (Masimo Corporation, Irvine, Calif.) may be used at the earlobe. Alternatively, a finger sensor such as a Nellcor™ Durasensor™ DS-100A Finger Sensor may be used at the index fingers.

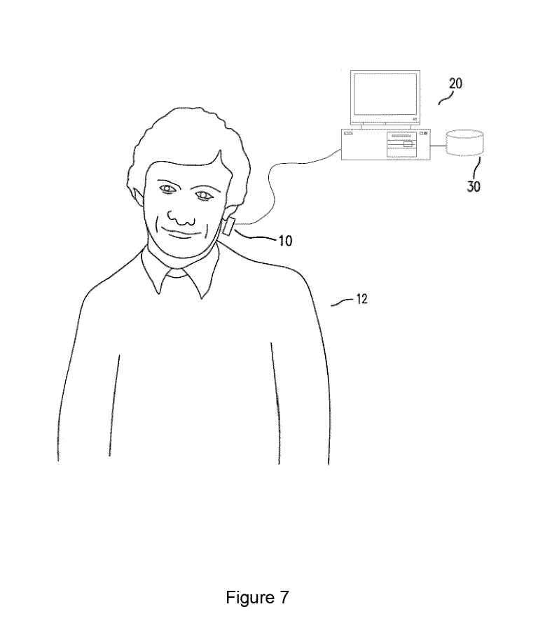

As shown in FIG. 7, a plethysmographic sensor 10 is applied to an earlobe of a subject (or patient) 12. The infrared outputs of the sensors are fed into a computer 20 equipped and configured to monitor the output of the outputs of the sensor 10 with linear AC differential amplifiers of the invention, the amplifiers are adjusted to give a +/−2.5 volt output to an analog-to-digital converter (for example, an A/D type USB-1208FS, manufactured by the Measurement Computer Corporation). The input signal is set to +/−1.0 volt. The pulse outputs from the sensors are digitized at 2 kHz with a 12 bit resolution.

Using a numerical computing application, such as MatLab™ (Version 7 3.0 0.267 (R2006b)), running on the computer (e.g., a computing device comprising at least a processor, memory/data storage means 30, a data bus, and input/output devices such as a keyboard, display screen, and a communications interface for communicating with the sensor 10), the inventive method, as implemented by a software program running on the computer hardware, processes the pulse signals using digital signal processing and statistical processing.

The time series data is first filtered using a digital filter to remove low frequency noise. In an exemplary embodiment of the invention, a Butterworth 4-pole filter with a low frequency cut-off at 0.7 Hz is used for removing low frequency noise caused by breathing effects, instrumentation noise, ambient light, RF signals, and motion.

Each pulse is processed with a Fast Fourier Transformation (FFT) and the peak magnitude at the fundamental frequency is obtained. A running standard deviation of typically 10 digitally filtered pulse magnitude data is computed and subsequently normalized by dividing the standard deviation by the mean computed over the same sample length. Then, the same routine is repeated after advancing one pulse in the serial data stream.

$${C}_{v} = \frac{σ}{μ}$$

$$σ \text{:} Standard Deviation$$

$$μ \text{:} Mean$$

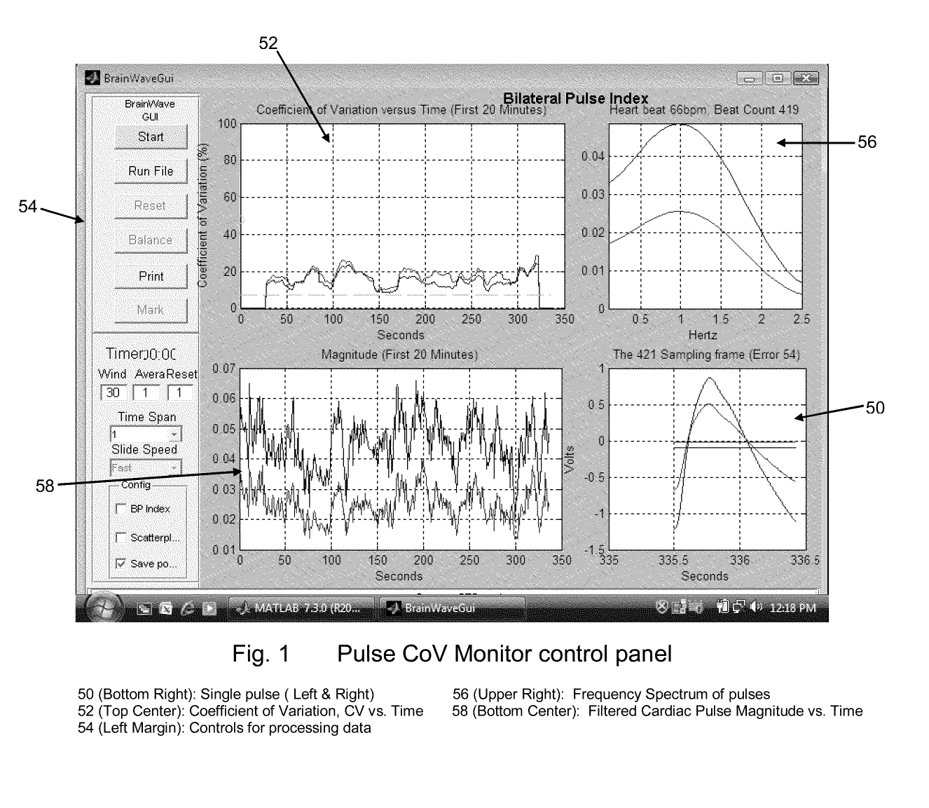

FIG. 1 shows an embodiment of a Pulse CoV Monitor control panel, operating under the Microsoft™ Windows™ computing environment, wherein four graphs and a control panel 58 are generated and displayed by the computer 20. In this embodiment, two signal inputs are processed corresponding to sensor devices 10 on the left and the right of the subject (e.g., at left and right earlobes or left and right fingers). Signals from the sensors 10 are receive by the computer 20 and processed as further provided below, and output is provided on the display screen of the computer 10. In the exemplary data shown in FIG. 1, the right signal is shown as a solid line and the left signal is a hatched line.

A first graph 50 at the bottom right shows a single pulse as generated by the two sensors. A second graph 52 at top center shows the CoV vs. time. A third graph 56 at the upper right plots the frequency spectrum of the pulses. A fourth graph 58 at the bottom center displays a filtered cardiac pulse magnitude vs. time. The control panel 58 at the left margin is provided as a graphical user interface (GUI) for controlling the monitoring of the sensors and display of the graphs.

In an implementation of the invention, the threshold noise level of the electronic system, the computer hardware, and software program without sensors and cables attached was determined to be 0.7% CoV rms. This was measured using both sine and saw tooth wave inputs from a signal generator. These waveforms were used to simulate pulse wave forms found in test subjects. The estimated noise base for our measurements is twice the threshold noise base, or 1.4% CoV rms. This value turned out to be well below the 4.0% CoV measured during sleep and anaesthesia experiments.

The inventor has observed that the standard deviation of the pulse signal at the earlobes and forehead decreased when the subjects were fully relaxed. The inventor has hypothesized that the standard deviation of the cardiac pulse signal will decrease when a subject is at rest and will further decrease when a subject is asleep.

Although the change in standard deviation can be used to track mental activities, the signals can be affected by skin pigmentation, sensor placement and shift in position. To overcome this difficulty, the inventor has normalized the standard deviation by dividing the standard deviation by the mean of the data computed over the same sample length as the standard deviation.

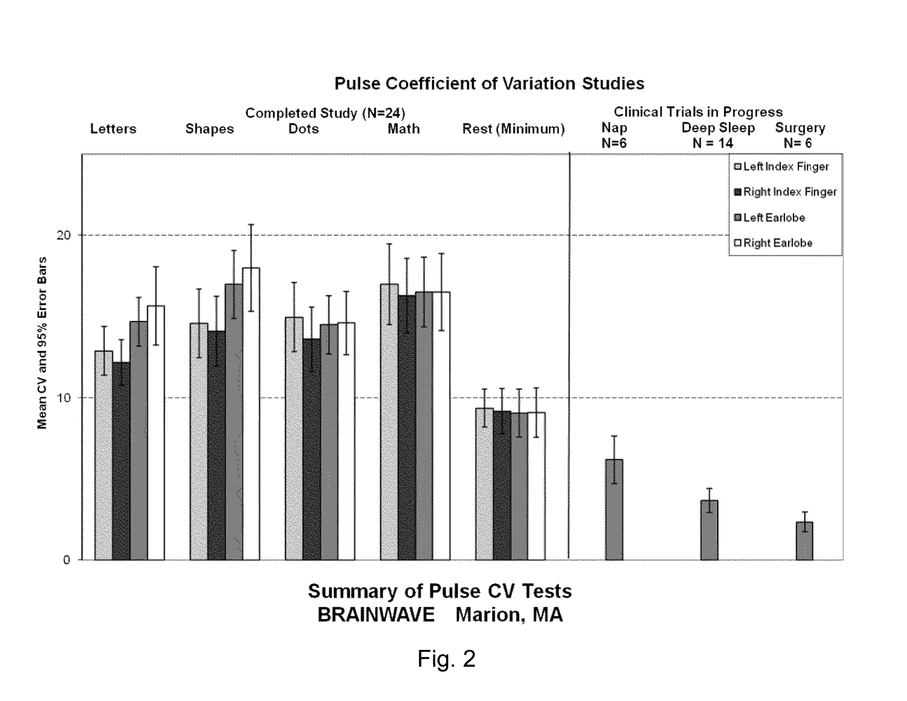

FIG. 2 shows the values of CoV obtained during various studies, including mental activity, rest, napping, deep sleep, and under anesthesia.

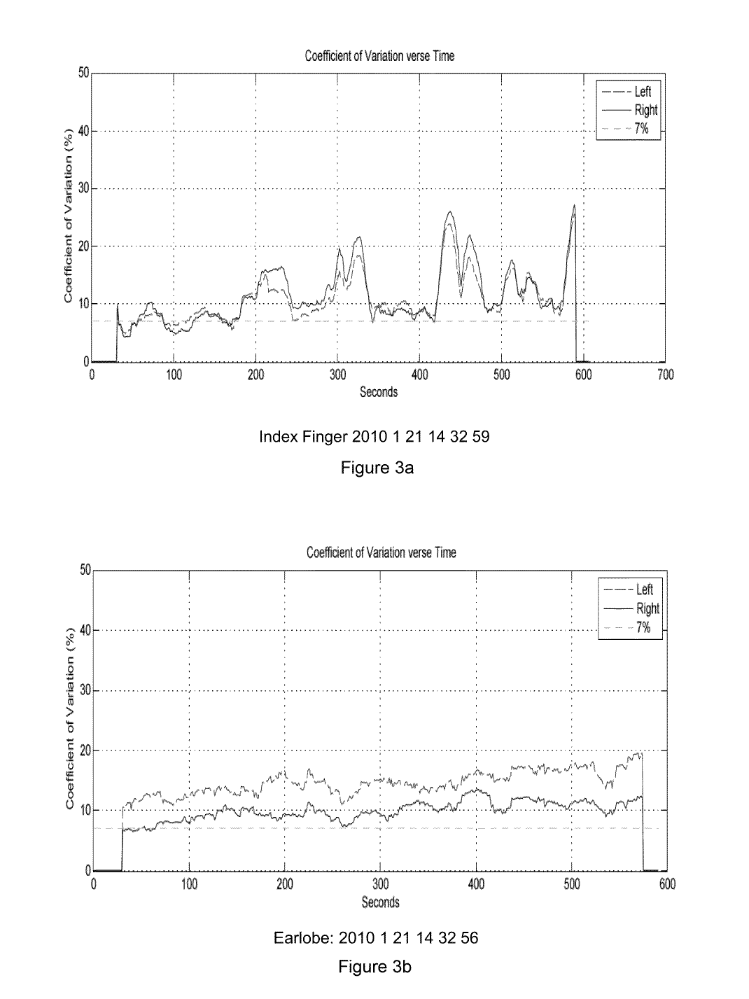

FIGS. 3a and 3b show the lower level of CoV noise obtained by monitoring at the earlobe (FIG. 3b) as compared to the index finger (FIG. 3a) during ten minute rest experiments.

A further feature of the invention is the measurement of breathing rate from the cardiac pulse.

When a subject inhales, the lungs expand and limit the chest cavity volume. This in turn limits the volume for the heart to expand during each cycle. The limited volume causes the blood pressure to increase during the pumping cycle. This produces a small variation in pulse magnitude which is normally buried in the cardiac pulse noise and not observable during casual inspection of the cardiac pulse magnitude after the FFT process has been carried out. This is especially apparent after using an AC coupled amplifier which introduces significant low frequency filtering in the breathing frequency spectral region.

An amplifier with a lower frequency response could be used to overcome this attenuation problem, but there would be considerable signal drift due to environmental effects. This would require automatic zero balancing of the DC amplifier to keep the pulse signal in the analysis range of the amplifier.

The signal has been measured to be as low as −50 dB below the peak pulse signal.

Average Respiratory Rates by Age
Newborns: 30-40 breaths per minute (0.5-0.66 Hz)
Less Than 1 Year: 30-40 breaths per minute (0.5-0.66 Hz)
1-3 Years: 23-35 breaths per minute (0.38-0.58 Hz)
3-6 Years: 20-30 breaths per minute (0.33-0.50 Hz)
6-12 Years: 18-26 breaths per minute (0.30-0.43 Hz)
12-17 Years: 12-20 breaths per minute (0.20-0.33 Hz)
Adults Over 18: 12-20 breaths per minute (0.20-0.33 Hz)

A breathing frequency in the range of 0.18-0.70 Hz is typically used in the breathing analyses of the cardiac pulse. This low level signal can be reliably detected when, instead of using the FFT, a spectral analysis program is used which incorporates statistical analysis to identify significant frequency bands.

The breath-rate monitoring software, executed on computer hardware, is based on modern spectral estimation theories. Although specific methods of spectral estimation and peak detection are employed, the present invention is for the basic discovery of the breath-rate as a spectral component of pulse CoV signal, regardless of the methods or techniques by which the observations are derived.

Spectral estimation theory is broadly divided into two main categories, parametric methods and non-parametric methods.

The non-parametric methods of spectral estimation such as the average periodogram, the discrete-time Fourier transform and the discrete-time discrete-frequency Fourier transform and the fast Fourier Transform and others are model independent and most suitable for large records of sampled data.

Parametric methods such as maximum-likelihood estimation, MUltiple SIgnal Classification (MUSIC), minimum variance spectral estimation (MUSE), modified Yule-Walker equation method (MYWE) and maximum entropy spectral estimation (MESE) and others are model dependent and best suited for short records of sampled data.

One aspect of the method of spectral estimation used in the present invention is maximum entropy.

Maximum Entropy Spectral Estimation (MESE), as noted previously, is a parametric method and is suitable for the short data records associated with the invention.

The MESE estimates the coefficients of an autoregressive model (AR) based on the principle of maximum entropy. The principle of maximum entropy estimation seeks estimates, AR-coefficients in this invention that maximize the randomness in the unknown data.

Generally, the random process from which the spectral estimate is to be obtained is assumed to be Gaussian. Given data from which a sample autocorrelation function can be estimated, the AR coefficients are estimated that best match the sampled autocorrelation so that the entropy per sample is maximized. By maximizing the entropy or randomness, minimum constraints are imposed on the data and minimal bias is introduced. Thus, the breathing cycle becomes apparent and accurately measurable.

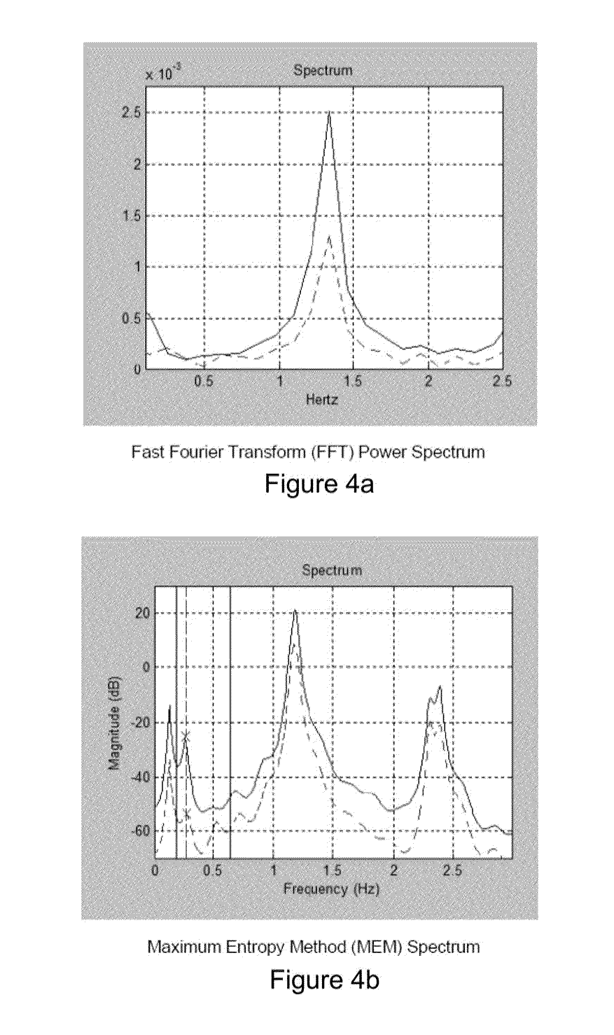

A chi-square test is used on the AR coefficients to detect and locate spectral peaks from the noise floor. In FIG. 4b the first such spectral peak in the frequency band from 0.18 to 0.70 Hz corresponds to the breathing rate. The large peak at about 1.0 HZ is the fundamental frequency of the cardiac pulse. This peak is about 50 dB above the breathing peak.

FIG. 4a shows the output from pulse data when using the FFT. There is no indication of a breathing signal in the frequency range between 0.18 to 0.704 Hz. The dual trace is from the output of a dual channel Pulse CoV monitor. FIG. 4b shows the MESE spectral peaks for the same data.

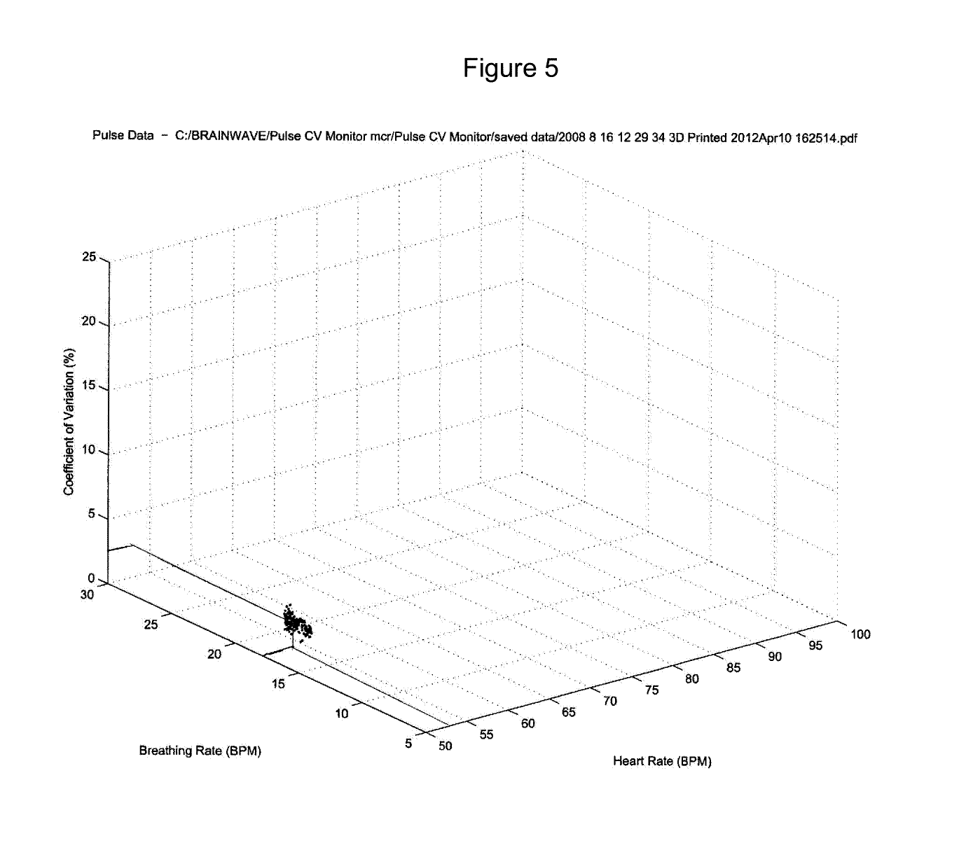

FIG. 5 shows a three dimensional plot of pulse coefficient of Variation (CoV) (Vertical Axis), Heart Rate (X Axis), and Breathing Rate (Y Axis). This type of graphic presentation allows the display of the output parameters from the subject invention as one display. Typically, to give a real time display, 60 pulses of data are displayed at a time. Then the oldest data point is removed from the display and the newest data point is added to the display. This type of display allows observation of the output data with a single glance at the data screen.

For example, when a subject is nearing deep sleep, data points of Coefficient of Variation (CoV), Heart Rate, and Breathing Rate will cluster in the lower left corner of the display. This grouping of data is referred to as the Comfort Corner for sleep.

An exemplary sequence of steps for carrying out the inventive method will now be described.

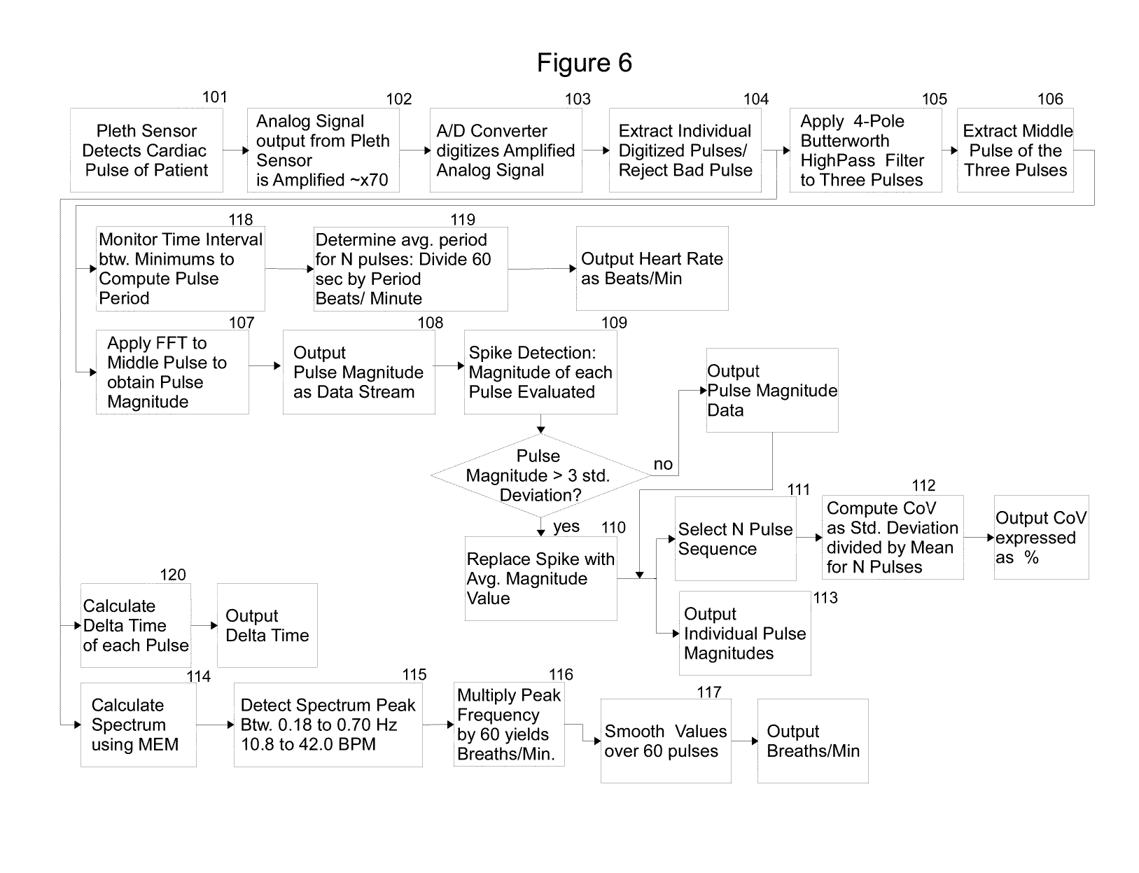

As shown in FIG. 6, the cardiac pulse is detected at step 101 with a device such as an optical plethysmographic sensor or an equivalent device for detecting a patient's pulse. Such a sensor may be located, for example, on the earlobe, forehead, or finger of the patient.

The analog signal generated by the sensor is amplified at step 102 in a linear amplifier with a band pass from 0.28 to 7.5 Hz. The gain of the amplifier is variable but is typically ×70, and the average output of the amplifier is in the range of +/−1.0 volts. At step 103, the output of the amplifier is fed to an analog-to-digital (A/D) converter with an input of +/−2.5 volt and 12 bit resolution.

At step 104, the pulse is extracted using minimum detection at the trough of the waveform. Where a pulse is measured as being unresolvable using minimum detection timing methods or exceeding the +/−2.5 volt range of the detection system, the pulse is rejected.

In step 105, three pulses in sequence are fed to a 4-pole Butterworth high-pass filter with a frequency cut off at 0.7 Hz. Three pulses are used to provide a long enough stream of data for the filter to respond fully and provide a reliable filtering of the middle pulse. At step 106, a middle of these three pulses is extracted using minimum detection. This middle pulse is then processed at step 107 using Fast Fourier Transform (FFT), and the magnitude at the peak of the spectrum is extracted at step 108. This output is referred to as the “pulse magnitude”.

At step 109, the magnitude of the pulse is evaluated by computing the standard deviation of pulses in future time. For example, 10 pulses are used when N is set to 10. If the current pulse is less than 3 standard deviations, it is accepted. If it is greater than 3 standard deviations, it is rejected. If the pulse is rejected, an average pulse is inserted into the data stream. This pulse is obtained by averaging the previous pulse before the rejected pulse with the pulse directly after the rejected pulse.

At step 111, the user is prompted to enter the number N, over which the computation of the Coefficient of Variation, CoV, is to be calculated. The CoV is defined as the standard deviation divided by the mean over N pulses. CoV is expressed as a percentage. This is a normalized value. Thus, the CoV is a universal descriptor and can be used between subjects without any corrections or rescaling. At step 112, the CoV is calculated over N pulses. Typically, 10 pulses are used for the computation. This value is output from the software in order to determine awareness.

At step 113, individual pulse magnitudes are provided as output.

Following a different path from the output of the A/D converter, a Maximum Entropy Method is applied at step 114 to process the time series. Typically, 60 seconds of pulses are analyzed. The frequency range from 0.18 to 0.70 Hz is evaluated for a maximum value (Spectral peak). (This band of frequencies represents the breathing frequencies which modulate the cardiac pulse).

At step 115, the peak of the spectrum is detected. At step 116, the frequency of the peak value is multiplied by 60, thereby to yield Breaths per Minute. At step 117, the values are smoothed with a digital filter and entered into a data file along with the original unfiltered values.

Step 118 takes place as another path from the step 106 wherein the middle pulse of three pulses is output. From this output, a pulse period is computed using minimum detection, by monitoring the time interval between successive minimums. From this, at step 119 an average period for N pulses is determined and by further dividing 60 seconds by the period to obtain an average pulse range in Beats per Minute. Using successive pulse minimum data, a delta time (period) is determined for each pulse at step 120.

Below, a number of additional exemplary practical applications for inventive Pulse CoV measurement are discussed.

1. Blood Pressure Measurement.

The measurement of blood pressure with a pressure cuff, technically called a sphygmomanometer, is often complicated by the anxiety state of the test subject. When a patient is first measured, the blood pressure will probably be higher than normal. As the patient sits and relaxes, the systolic/diastolic blood pressure drops until a normal pressure is reached. There is no good means for a doctor or practitioner to know the state of anxiety of the patient. The measurement of Pulse CoV provides such a measurement. The incorporation of Pulse CoV measurement into conventional pressure cuffs would provide this anxiety measurement in a reproducible and recordable form and speed up this measurement in the clinical setting.

2. Anesthesia Drug Administration.

The pulse CoV measurement can be used for control of anesthesia drug administration such as self-administration during child birth. As various drugs are administered to the patient, the level of alertness to stimulus can be monitored. The concept of continuously monitoring of the patient is to determine when the level of alertness has bottomed out or minimized.

If this information is obtained, the administration of drugs can be stopped to reduce overdosing, increased probability of sickness from the anesthesia, and reduce recovery times from excessive drug doses.

The method can be used in a closed loop feedback system to control anesthesia drug administration. This use can be employed by the anesthesiologist when a patient is permitted to self-administer the drug. The feedback control system can anticipate over dosing by the patient and provide a more uniform and controlled anesthesia than to just allow the patient to self-administer. This would provide an overriding of the patient's commands.

3. Sleep Detection.

The Pulse CoV method can be used for sleep detection, e.g., of aircraft ground controllers, pilots and other persons involved in the critical operation of vehicles where safety is of utmost importance.

In these applications the measurement of the Pulse CoV can either alert the subject of oncoming sleep, or remove the subject from control of the system. If the sensor is installed before operation of the system, the system can determine if an alert individual is at the controls before the equipment can be operated.

4. Brain Activity.

The measurement of CoV represents a measure of brain activity. This is a useful measurement for computer operators to compute the work load for the subject. By integrating the CoV measurement over a period of mental work, the measurement can be used to prevent fatigue and subsequent health problems.

5. Biofeedback Therapy.

The monitoring of CoV can be used for biofeedback therapy. This can be implemented using, e.g., a personal computer or other suitable computing hardware. The subject would install the program software and use the CoV measurement as a means to enhance relaxation, and a state of well-being.

6. Awareness Monitoring During Anesthesia Use.

The Pulse CoV method can be incorporated into BiSpectral (BIS) Index analyses to broaden the scope of awareness monitoring during anesthesia use. BiSpectral (BIS) Index analyses does not provide a continuous monitoring output from conscious to fully unconscious states. Pulse CoV does. When these methods are combined, a better measure of awareness will be obtained with a quality control check of the BIS measurement.

7. New defibrillator designs now incorporate oximeters into the product to determine blood oxygen levels. The Pulse CoV method can also be incorporated into defibrillators to provide awareness and breathing rate measurements to further aid in determining the subject's physical condition.

The foregoing description of the present invention, and the Figures to which the description refers, are intended as examples only, and are not intended to limit the scope of the invention. It is anticipated, for example, that one skilled in the art will likely realize additional alternatives that are now apparent from this disclosure. Accordingly, the scope of the invention should be determined solely from the following claims and limitation should be inferred by the foregoing description or the Figures.

I claim:

1. A method of measuring awareness in a human subject, comprising the steps of:
using a sensor and cardiac pulse measurement hardware, measuring a cardiac pulse at a location on the subject, the measurement providing pulse signals;
using a computer apparatus, applying a method to the pulse signals comprising eliminating noise from the pulse signals by using an error detecting algorithm to remove high magnitude pulse noise and retain lower magnitude data before the Coefficient of Variation (CoV) is calculated, and applying the pulse Coefficient of Variation (CoV) method to the measured pulse signals, to obtain Coefficient of Variation (CoV); and
evaluating the awareness of the subject based on the retained low Coefficient of Variation (CoV) data,
wherein high amplitude Coefficient of Variation (CoV) is indicated as greater than 7% CoV, and
wherein low Coefficient of Variation (CoV) data is indicated as less than 7% CoV.

2. The method of claim 1, wherein,
in said measuring step, the sensor is a single cardiac pulse sensor placed at one of i) a forehead of the subject, ii) an ear canal of the subject, iii) an earlobe of a subject, or iv) a nose of the subject, v) a finger of the subject vi) a toe of a subject and
the sensor is one of i) a plethysmographic sensor, and ii) a pulse oximeter sensor.

3. The method of claim 1, wherein,
the computer apparatus, in eliminating the noise from the pulse signals by using the error detecting algorithm to remove the high magnitude noise and retain the low magnitude data, obtains a filtered time series data by i) digitizing the pulse signals, ii) subjecting the filtered digitized pulse signals to statistical processing for the elimination of the noise, and
the pulse Coefficient of Variation (CoV) method is applied to the filtered time series data to obtain the retained Coefficient of Variation (CoV) data.

4. The method of claim 3, wherein,
the computer apparatus, in eliminating the noise from the pulse signals, applies a high pass digital filter on the time series data below 0.7 Hz to remove low frequency noise caused by breathing effects, instrumentation noise, ambient light, RF and motion.

5. The method of claim 1, wherein said step of applying the pulse Coefficient of Variation (CoV) method comprises use of BiSpectral (BIS) Index analysis technology, and
said step of evaluating the awareness of the subject includes monitoring subject awareness during surgery using both the BiSpectral method and the Coefficient of Variation (CoV) Method.

6. The method of claim 1, wherein the evaluating step is used to determine whether the subject has attached the sensor prior to operating a mechanical system and an electronic system to measure a parameter of the subject.

7. The method of claim 1, wherein the step of evaluating the awareness of the subject is included in a feedback loop controlling administration of drugs to the subject.

8. The method of claim 1, wherein,
the sensor is integrated as part of a pressure cuff for measuring the blood pressure of the subject, and
said step of evaluating the awareness of the subject is performed to evaluate a level of anxiety of the subject, and blood pressure readings are obtained by way of the pressure cuff upon a determination from the evaluated anxiety of the subject that the subject is in a relaxed state.

9. The method of claim 1, wherein,
the subject is a computer operator,
said measuring step is conducted over a work period, and
said step of evaluating the awareness of the subject evaluates a work load on a machine from the operator by summing retained Coefficient of Variation (CoV) data over the work period.

10. The method of claim 1, wherein,
the sensor is one of a plethysmographic sensor or an oximeter sensor,
the sensor comprises a motion sensing element configured to detect motion, the motion sensing element providing a motion signal, and
the method comprises a further step, prior to the step of applying the pulse magnitude method error detection, of rejecting noise based on the motion signal.

11. The method of claim 1, comprising the further step, based on the retained Coefficient of Variation (CoV) data, of making a visual representation on a display device of the computer apparatus wherein the retained Coefficient of Variation (CoV) data is plotted on a graph against corresponding heart beat data, and breathing rate data.

12. The method of claim 11, wherein,
is the retained Coefficient of Variation (CoV) data, the heart beat data, and the breathing rate date is represented as a three-dimensional plot, and
the three-dimensional plot is continuously updated to present real time data, the three-dimensional plot being updated regularly after a predetermined number of pulses.

13. The method of claim 12, wherein,
the three-dimensional plot uses a color change to represent a pulse magnitude value and thereby provide a four-dimensional plot in real time.

14. The method of claim 1, wherein,
the sensor is one of a plethysmographic sensor or an oximeter sensor,
the sensor comprises an ambient light sensing element configured to detect ambient light of a wavelength corresponding to a wavelength sensed by the sensor, and
the step of using this information for rejecting ambient light noise before selecting pulse data for further processing using CoV computation.

15. The method of claim 1, wherein,
output from the sensor and the pulse measurement hardware is digitized and pulse signals are filtered by a 4-pole high pass filtering with a frequency cut-off at 0.7 Hz, and
the pulse Coefficient of Variation (CoV) method is applied to the further filtered digitized pulse signals to obtain the retained Coefficient of Variation (CoV) data.

16. The method of claim 1, wherein,
the pulse signals are analyzed by a spectral statistical analyzer, operating on the computer apparatus, to generate a statistical enhanced spectrum of the pulse signals in the frequency domain, and a breathing rate is determined from peaks detected from said statistical enhanced spectrum, and
said step of evaluating the awareness of the subject is further based on said determined breathing rate.

17. The method of claim 16, wherein said spectral analyzer applies a time domain to frequency domain computer program to extract signals corresponding to breathing from the pulse signals in the frequency range of 0.18-0.70 Hz.

18. The method of claim 16,
wherein said spectral statistical analyzer applies maximum entropy spectral estimation (MESE) to estimate coefficients of an autoregressive model (AR) to extract signals corresponding to breathing from the pulse signals, and
wherein said determined breathing rate is determined by analyzing spectral content of the pulse signals using spectral analysis with statistical enhancement in the frequency range of 0.18-0.70 Hz.

19. The method of claim 16, wherein,
a spectral analysis for determining the breathing rate is enhanced by detecting errors caused by harmonics of a breathing fundamental to prevent tracking of incorrect spectral peaks.

20. A device for implementing a method of measuring awareness in a human subject, comprising the steps of:
a sensor and cardiac pulse measurement hardware configured to measure a cardiac pulse at a location on the subject, the measurement providing time domain pulse signals; and
a computer apparatus operatively connected to the sensor by way of an input/output communications bus, the computer apparatus further comprised of a processor, memory, and an information storage facility having stored therein software executable to cause the computer to
A) transform the time domain pulse signals to a frequency domain, including i) extracting magnitudes of dominant pulse frequencies to obtain pulse data (107), ii) computing a running standard deviation of the magnitudes of the dominant pulse frequencies (112), iii) dividing the computed running standard deviation by a corresponding moving average to obtain the pulse coefficient of variation (112), and iv) generate a statistically enhanced spectrum of the pulse signals in the frequency domain,
B) evaluate the awareness of the subject based on the obtained pulse coefficient of variation, and
C) evaluate a breathing rate of the subject from peaks detected from said statistically enhanced spectrum, thereby to further evaluate the awareness of the subject.
